(19) United States
(12) Patent Application Publication
(10) Pub. No.: US 2026/0294419 A1
(43) Pub. Date: Oct. 1, 2026

(54) PRESSURE SENSING SELF-RETAINING RETRACTOR

(71) Applicant: ARIZONA BOARD OF REGENTS ON BEHALF OF THE UNIVERSITY OF ARIZONA, Tucson, AZ (US)

(72) Inventors: David MARGOLIS, Tucson, AZ (US); McKenzie BIEG, Tucson, AZ (US); Mark BOSSET, Tucson, AZ (US); Dania PEREZ, Tucson, AZ (US); Timor SHAHIN, Tucson, AZ (US); Evan SMITH, Tucson, AZ (US); Urs UTZINGER, Tucson, AZ (US)

(21) Appl. No.: 19/479,671

(22) PCT Filed: Apr. 24, 2024
(86) PCT No.: PCT/US2024/025983

§ 371 Date: Oct. 28, 2025

Related U.S. Application Data
(60) Provisional application No. 63/463,141, filed on May 1, 2023.

(51) Int. Cl. A61B 17/02; A61B 17/00; A61B 90/00

(52) U.S. Cl. CPC A61B 17/02; A61B 2017/00119; A61B 2090/064; A61B 2090/0813

(57) ABSTRACT

A surgical self-retaining retractor system includes a self-retaining retractor having first and second tissue engaging members that oppose one another and maintain an opening in an incision. First and second force sensors are respectively coupled to the first and second tissue engaging members such that the force sensors detect force being exerted thereon by tissue when the self-retaining retractor is maintaining an opening through the incision.

### CROSS REFERENCE TO RELATED APPLICATION

This application claims the benefit of U.S. Provisional Application Ser. No. 63/463,141, filed May 1, 2023, the contents of which is incorporated herein by reference.

### BACKGROUND

Surgical retractors are well-known tools in the operating room, playing a crucial role in maintaining tissues and organs away from the surgical site by separating the edges of an incision or wound for a maximal field of view. Surgical retractors are available in various shapes, sizes, and styles, providing both handheld and self-retaining options designed for specific surgical needs. Conventional retractors have blades that typically are at a right angle and can be smooth, raked, or hooked, and can have removable or fixed prongs or tines. Surgical retractors are made from biocompatible materials that can be placed in an autoclave to be sterilized so they may be reused for multiple, successive surgical procedures. A common material used in the making of retractors is stainless steel. Stainless steel is preferred because of its strength and its ability to be sterilized via autoclave. Other materials that are sometimes employed include titanium, carbon fiber, and silicone. Existing self-retaining retractors, such as Meyerding, Markham-Meyerding, Gelpi, Cerebellar Jansen, Weitlaner, and Williams, are commonly utilized due to their solid stainless steel construction and efficient self-locking mechanisms. While retractors are key instruments in surgery, the disadvantages of such retractors become clear during longer operations, during which patients with prolonged use have a higher chance of experiencing chronic neuropathic pain due to soft tissue trauma. There is a need for surgical retractors capable of sensing force against the tissue interface to elevate post-operative patient rehabilitation.

### SUMMARY

When performing surgical procedures, surgeons will commonly use self-retaining retractors to facilitate visualization of the surgical field without the need for an assistant to retract the soft tissues. One potential complication associated with the use of self-retaining retractors is the potential for the retractor tines to apply excessive pressure to the soft tissues over prolonged periods. This can lead to intra-and post-operative problems, particularly nerve palsies that can cause pain and functional problems for patients.

In one aspect, a self-retaining retractor is described herein that can measure pressure at the retainer-tissue interface. This equipment can then alert the surgeon if excessive pressure is applied to minimize the chances of irreversible soft tissue trauma.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows an isometric view of one example of a self-retaining surgical retractor.

FIG. 2 shows a top view of one example of a self-retaining surgical retractor.

FIG. 3 shows a simplified block diagram of one example of the overall pressure-sensing self-retaining retractor system.

FIG. 4 shows an isometric exploded view of one example of a self-retaining surgical retractor with sensors and a backplate.

### DETAILED DESCRIPTION

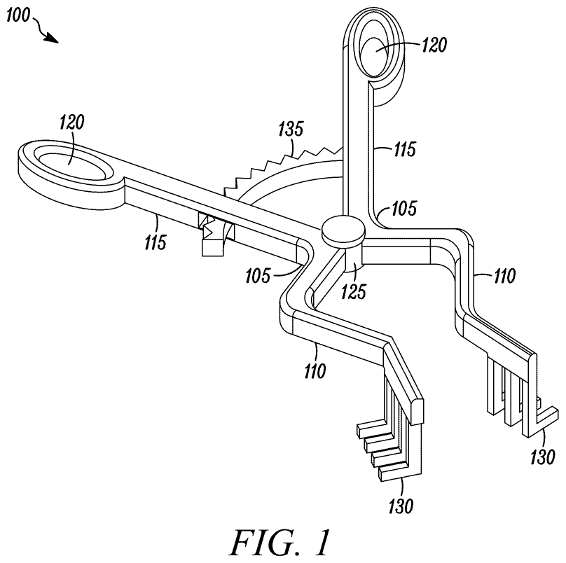

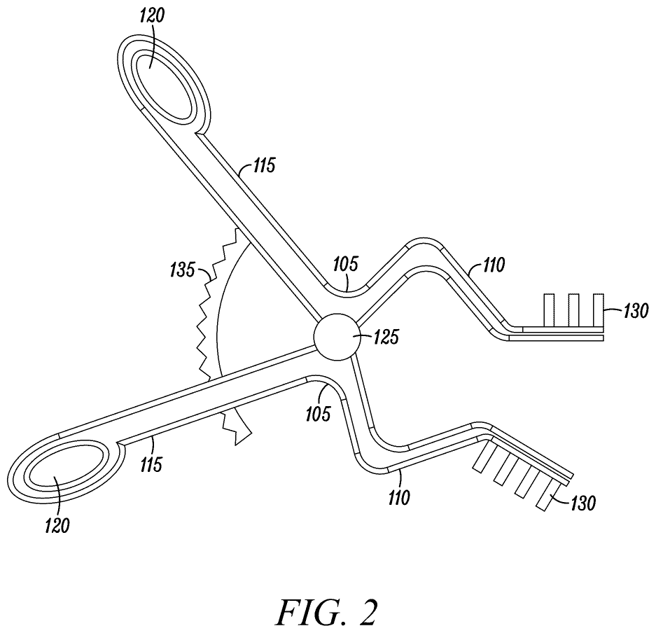

FIG. 1 shows an isometric view and FIG. 2 shows a top view of one example of a self-retaining surgical retractor 100 (e.g., a handheld surgical retractor) that may be provided with a pressure sensing mechanism as described herein. For purposes of illustration, the surgical retractor 100 will be described as a Weitlaner retractor. More generally, however, the present disclosure applies to any type of self-retaining surgical retractor. The surgical retractor 100 includes two spreader levers 105 which are connected in a hinged manner by a hinged connection 125. Each of the two spreader levers 105 includes a spreader arm 110 positioned on one side of the hinged connection 125 and an actuation arm 115 positioned on the other side of the hinged connection 15. The proximal end portions of the two actuation arms 115 are ergonomically designed for easy grip and manipulation. For instance, in this example, the actuation arms 115 are each provided with finger insertion loops 120 serving to move the spreader levers 105 manually and to bring the spreader arms 110 into various pivoting or spreading positions. The surgical retractor 100 may be formed of any material suitable for use at a surgical site such as, for example, stainless steel or the like.

The hinged connection 125 may include a sleeve having an enlarged head. The sleeve extends through one side of an opening formed in the spreader levers 105. The sleeve has an opening to receive a fixation element (not shown) on the other side of the opening formed in the spreader levers 105. In some embodiments, the sleeve may include internal threads and the fixation element may be a screw having external threads corresponding to the internal threads of the sleeve. In some embodiments, the fixation element may alternatively be bonded to the sleeve in any conventional manner such as, for example, welding, adhesives, etc.

The distal end portions of the spreader levers 105 include tissue-engaging members 130 that ensure tissue retraction without causing damage. In the example illustrated, the tissue-engaging members are a plurality of prongs or tines. The tines may be sharp, tapered, and slightly curved to facilitate easy insertion and retraction of tissue. More generally, the tissue engaging members 130 in various embodiments of the self-retaining retractor may have a wide variety of different configurations to accommodate different surgical sites and different anatomies.

The self-retaining surgical retractor 100 includes an integrated locking mechanism 135 that securely lets the surgeon position the retractor within the surgical field. The locking mechanism 135 may utilize a ratcheting system or a cam mechanism to provide incremental adjustments and maintain the desired degree of tissue retraction. Additionally, the locking mechanism 135 may feature a quick-release mechanism to facilitate rapid removal of the retractor once the surgical procedure is completed.

During operation, the surgeon positions the self-retaining retractor 100 within the surgical field and adjusts the positions of tissue-engaging members 130 (e.g., tines) to achieve optimal tissue exposure. Once the desired configuration is achieved, the locking mechanism 135 is engaged to secure the retractor in place. Throughout the procedure, the device maintains consistent retraction, allowing the surgeon to focus on the operative task without requiring continuous manual manipulation.

As previously mentioned, during surgical procedures, self-retaining retractors are used to hold the tissue open to access and visualize the working field without the need to hold the tissue open manually. However, problems arise when excessive pressure is applied to nervous and vascular tissue, causing them to be compressed at high forces for prolonged periods of time, resulting in pressure injuries. To address this problem, force sensors are mounted to the tissue-engaging members of the self-retaining retractor 130 (e.g., tines). The use of such force sensors to monitor the magnitude of the pressure and the time under pressure of the tissue can allow surgeons to mitigate these potential postoperative complications.

The force sensors that are incorporated into self-retaining retractors as described herein may employ any suitable force sensing technology including, without limitation, load cells, piezoelectric, piezoresistive, and capacitive sensing technologies. In some particular embodiments, the force sensors may be thin, flexible force sensors in which a thin film of pressure-sensitive material is located between a pair of conductors. Such flexible force sensors are commercially available. For example, force sensors in the form of pads or strips are available from Tekscan, Inc., which provides Flexforce™ thin film sensors with thicknesses in the range of 0.2 mm.

One or more force sensors such as those described above may be applied to the tissue engaging members 130 on one or both of the spreader levers 105. The force sensors may be attached to the tissue engaging members 130 in any suitable manner that allows the force and/or pressure exerted by the tissue to be accurately measured. The attachment mechanism may be chosen based on the particular configuration of the tissue-engaging members and the force sensors that are chosen. For instance, in some embodiments that employ tines or the like, a force distribution member such as a flat rigid plate or other substrate may be secured to the tines and the force sensor may be mounted on the plate or substrate. In this way, the force of the tissue may be more uniformly distributed over the sensors. The plate or substrate may or may not be formed from the same material as the retractor. For example, in some cases, the plate or substrate may be formed from stainless steel. In some embodiments, the force sensors may be permanently attached to the tissue-engaging members, or they may be attached in a removable manner. For example, in some cases, the plate or substrate and the force sensors may be secured in place using a highly heat-resistant adhesive such as medical-grade epoxy.

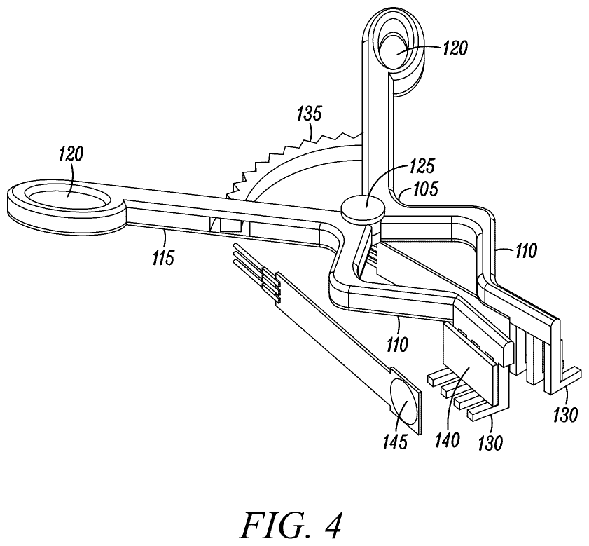

FIG. 4 shows an isometric exploded view of one example of the self-retaining surgical retractor that includes force sensors and a backplate. In this example the self-retaining surgical retractor is of the type shown in FIGS. 1 and 2. In FIGS. 1, 2 and 4, like references are denoted by like reference numerals. As shown in FIG. 4, a backplate 140 is secured to the tissue engaging member 130 and a force sensor 145 is mounted on the backplate 140. While only a single force sensor 145 is being shown, in other embodiments force sensors may be located on both tissue engaging members 130.

The force sensors mounted to the retractor communicate with a processing unit over a wired or wireless connection. The processing unit receives (e.g., analog) signals from the force sensors and processes the signals to generate force or pressure data that can be displayed to allow the surgeon to monitor the magnitude of the pressure and the time over which the tissue is under pressure and thereby mitigate potential postoperative complications. In some embodiments, the output signals from the force sensors are output voltages that are converted to pressure values using a previously established calibration curve.

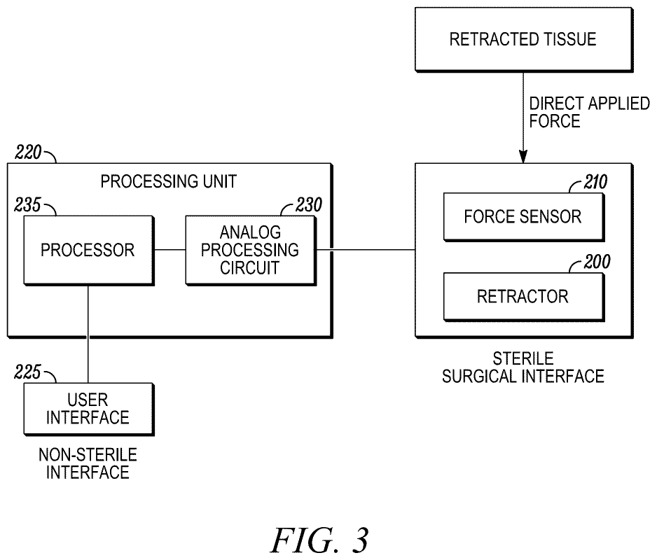

FIG. 3 shows a simplified block diagram of one example of the overall pressure-sensing self-retaining retractor system. The system can be divided into those components that are to be located in the sterile field of the surgical space and those components that may be located in a non-sterile field. As shown, the components in the sterile field include the self-retaining retractor 200 and the force sensor(s) 210 onto which force is applied by the retracted tissue 215. The remaining components may be located in the non-sterile field and include the processing unit 220 and a graphical user interface 225. The processing unit 220 includes an analog processing circuit 230 that receives the signal from the force sensor(s) and converts it to a digital signal that is sent to a processor 235, which converts the digital signal to a pressure or force value.

The value is transmitted to the graphical user interface (GUI) 225, featuring a display that showcases pressure or force values alongside additional parameters like the duration since force sensor activation and instances where the force surpasses one or more thresholds. In some embodiments, the graphical user interface alerts the user if predetermined pressure or force thresholds are exceeded for a predetermined period of time. The alert that is generated may be audible and visual. The time and pressure or force thresholds may be user-selectable and may be entered on the graphical user interface 225 (e.g. using a touchscreen, dedicated buttons, etc.) In some embodiments, the processing unit 220 may be a portable unit that is battery-powered. The graphical user interface 225 may be any suitable display device and in some embodiments may be a laptop computer, tablet, smartphone or the like. The processing unit 220 and the graphical user interface 225 may communicate over a wireless link (e.g., Bluetooth, Wi-Fi). In yet other embodiments, the processing unit 220 and the graphical user interface 225 may be provided as a single unit.

When the force sensor(s) 210 communicates with the processing unit 220 over a wired connection, the wires from the force sensor(s) should be encased in a sterilizable material. For instance, the wired connection might utilize silicone-insulated wires with one end connected to the force sensor(s) 210 and a removable connector at the opposite end, which can be connected to the processing unit 220.

In one illustrative example, the operation of the pressure-sensing self-retaining retractor system described herein may proceed as follows. A sterile operator will unpack the sterile system and give the connector from the force sensor(s) to a non-sterile operator, who will plug the connector into the processing unit and set pressure and time thresholds using the graphical user interface. The surgeon will then conventionally use the retractors. The patient's tissue will then be exerting pressure on the force sensor(s), which changes the voltage output by the sensor(s). The voltage output is sent to the processing unit, which converts the voltage to a digital signal and uses a calibration curve to calculate the applied force. The pressure applied to the force sensor(s) by the patient's tissue is then calculated from the applied force. The processing unit then records the pressure and the time and sends the pressure value with the time to the graphical user interface to be displayed. An alarm may be triggered if the pressure remains above the pressure threshold for a time that exceeds the time threshold.

Although the present disclosure has been described regarding Weitlaner self-retaining retractors, more generally the pressure sensing techniques described herein are also applicable to other types of self-retaining retractors including, without limitation, Meyerding, Markham-Meyerding, Gelpi, Cerebellar Jansen, and Williams retractors. In yet other embodiments, the pressure sensing techniques described herein are also applicable to custom designed self-retaining retractors that in one or more aspects are configured differently from conventional types of self-retaining retractors.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described to best explain the principles of the embodiments and their practical applications, thereby enabling others skilled in the art to best utilize the embodiments and various modifications as may be suited to the particular use contemplated. Accordingly, the present embodiments are to be considered illustrative and not restrictive, and the invention is not to be limited to the details given herein but may be modified within the scope and equivalent of the appended claims.

1. A surgical self-retaining retractor system, comprising:
a self-retaining retractor having first and second tissue engaging members that oppose one another and maintain an opening in an incision; and
first and second force sensors respectively coupled to the first and second tissue engaging members such that the force sensors detect force being exerted thereon by tissue when the self-retaining retractor is maintaining an opening through the incision.

2. The surgical self-retaining retractor system of claim 1, wherein the first and second tissue engaging members respectively include a first and second series of blades or tines.

3. The surgical self-retaining retractor system of claim 1, wherein the self-retaining retractor and the first and second force sensors are sterilizable.

4. The surgical self-retaining retractor system of claim 1, further comprising a processing unit in communication with the first and second force sensors for receiving signals therefrom and in response thereto generating an output reflective of the force and/or pressure exerted on the force sensors by the tissue.

5. The surgical self-retaining retractor system of claim 4, further comprises a wired connector having a first end connected to the first and second force sensors and a second end connectable to the processing unit, wherein the wired connector is sterilizable.

6. The surgical self-retaining retractor system of claim 4, wherein the processing unit includes a graphical user interface for setting a user-defined pressure threshold such that an alert is generated if the pressure exerted on the first and second force sensors exceeds the user-defined pressure threshold.

7. The surgical self-retaining retractor system of claim 1, wherein the self-retaining retractor is a Weitlaner retractor.

8. The surgical self-retaining retractor system of claim 2, further comprising first and second force distribution members extending over, and mounted, to the series of blades or tines in the first and second series of blades or tines, respectively, wherein the first and second force sensors are respectively connected to the first and second force distribution members.

9. A method of monitoring force and/or pressure being exerted by tissue exposed in an operative field in a patient upon tissue engaging members of a self-retaining retractor than maintains the tissue in place, comprising:
generating signals indicative of the force being exerted by the tissue on the tissue engaging members of the self-retaining retractor that maintains the tissue in place;
communicating the signals to a processing unit located in a non-sterile field external to a sterile field in which the self-retaining retractor is being used; and
generating an output from the signals communicated to the processing unit, the output being reflective of the force and/or pressure exerted by the tissue.

10. The method of claim 1 further comprises:
receiving user input specifying a pressure threshold; and
generating an alert if the pressure threshold is exceeded.

11. The method of claim 1, further comprises:
receiving user input specifying a pressure threshold and a time duration threshold; and
generating an alert if the pressure threshold is exceeded for a period that exceeds the time duration threshold.
